(19) United States
(12) Patent Application Publication
(10) Pub. No.: US 2008/0300468 A1
(43) Pub. Date: Dec. 4, 2008

(54) System and apparatus for inspection of feet

(76) Inventor: Albert MURILLO, Templeton, CA (US)

Correspondence Address: LAURA N. TUNNELL, P.O. BOX 6003, SANTA BARBARA, CA, 93160, US

(21) Appl. No.: 11/809,142

(22) Filed: May 30, 2007

(51) Int. Cl. A61B 19/00

(52) U.S. Cl. 600/247

(57) ABSTRACT

A collapsible apparatus operable for enabling a person to self-inspect the soles of the person's feet or for enabling another person to inspect the soles of the person's feet when the person is in a seated position. The apparatus includes bifurcated vertical walls comprising an upper portion and a lower portion wherein the upper and lower portions of the vertical walls are connected to one another by extendable means operable for enabling a person to conveniently transport the apparatus in a collapsed configuration and rapidly reconfigure the apparatus for use.

### BACKGROUND OF THE INVENTION

1. Field of the Invention

The present invention is in the area of medical equipment, and more specifically relates to apparatus and methods for inspecting feet.

2. Prior Art

Diabetics frequently experience diseases of the feet. Complications associated with diabetes such as blisters, bruises, rashes, in-grown toenails, etc on a diabetes victim will manifest themselves on the soles of a victim's foot. If these problems are not properly treated in time, these complications will exacerbate to the point where the foot becomes infected with gangrene. Once gangrene sets in and accumulates in one's foot, a doctor may have no choice but to amputate the gangrenous infected area in order to prevent its spread to the rest of the body. Unfortunately, many diabetes patients are elderly people who cannot properly examine the soles of their own feet. Such elderly people have less flexibility and are oftentimes overweight. Therefore, the only way to ensure proper inspection of their feet is to either have a doctor perform the inspection, or have a lay person inspect the person's feet. However, having other people inspect one's own feet may be awkward and embarrassing.

To overcome this self-inspection problem, the present inventor, in U.S. Pat. No. 7,137,950, has disclosed an apparatus that enables the self-inspection of a person's feet when the person is comfortably seated. While the apparatus disclosed in the '950 patent enables a person to perform its intended self-inspection function, the apparatus is bulky and inconvenient to transport. There remains a need for an apparatus operable for enabling the self-inspection of one's feet that is collapsible and easily transported.

### SUMMARY

The present invention is directed to a collapsible apparatus operable for enabling a person to view the solar surface of the person's feet. The collapsible apparatus substantially obviates one or more of the limitations of the related art. To achieve these and other advantages and in accordance with the purpose of the invention, as embodied and broadly described herein, in its most general form, the collapsible apparatus for inspecting feet comprises a top wall having a top mirror tiltably attached thereto, and a bottom wall having a left side mirror, a right side mirror and a center mirror attached thereto. A telescopically extendable locking means connects the top wall to the bottom wall. In operation, a person manually releases the telescopically extendable locking means and raises the top wall to a convenient height for viewing the top hinged mirror. The extendable locking means is then locked in position to maintain the height separation between the bottom and top walls. The person then seats himself such that he/she can comfortably view the top mirror and places his/her feet above the center mirror on the bottom wall. An image of the plantar surface of the feet appears on the top mirror and enables the person to visualize a diseased condition on the bottom of the feet.

In another embodiment, the collapsible apparatus for self-inspecting feet is a transportable console comprising a bifurcated left vertical wall, a bifurcated right vertical wall, a top wall having a top mirror tiltably attached thereto, a bottom wall having a left side mirror, a right side mirror and a center mirror attached thereto, and telescopically extendable locking means connecting the top wall and the bottom wall. The apparatus further comprises feet holding bars, one end of which is attached to a lower portion of the left bifurcated vertical wall and the other end being attached to a lower portion of the right bifurcated vertical wall. The top wall and the bottom wall are connected to one another by telescopically extendable support rods.

The top mirror is affixed to a bottom side of the top wall via affixing means. The center mirror is affixed substantially in the center of the bottom wall via affixing means. The right side mirror is affixed to the bottom wall substantially adjacent to one edge of the center mirror via affixing means; and the left side mirror is similarly affixed to bottom wall substantially adjacent to the opposing edge of the center mirror via affixing means. The foot supporting bars provide means for comfortably supporting the feet above the center mirror for viewing.

The features of the invention believed to be novel are set forth with particularity in the appended claims. However the invention itself, both as to organization and method of operation, together with further objects and advantages thereof may be best understood by reference to the following description taken in conjunction with the accompanying drawings.

### BRIEF DESCRIPTION OF THE DRAWINGS

FIG. 1 is a is a perspective view of an apparatus operable for enabling the self-inspection of a person's feet in accordance with the prior art.

FIG. 2 is a is a perspective view of an apparatus in accordance with the present invention operable for enabling the self-inspection of a person's feet, the apparatus illustrated in a fully extended configuration.

FIG. 3 is a is a perspective view of the apparatus operable for enabling the self-inspection of a person's feet of FIG. 2 shown in a collapsed configuration for compact storage or transport.

### DESCRIPTION OF THE PREFERRED EMBODIMENTS

In U.S. Pat. No. 7,137,950, the content and teaching of which is incorporated herein by reference thereto, the present inventor disclosed an apparatus operable for enabling the self-inspection of a person's feet. The foot inspection apparatus may be used by diabetic patients or others to search for blisters, bruises, rashes, in-gown toenails, red spots, etc. on the soles of their feet. The prior art apparatus is also designed to be used by caregivers such as physicians, podiatrists, nurses, physician's assistants etc. for the purpose of evaluating and diagnosing any foot-related maladies. The prior art apparatus disclosed in the '950 patent is designed to be used at home by people for the purpose of self-evaluation. The present apparatus is an improvement over the aforesaid prior art apparatus in that it is collapsible and can be conveniently stored and transported as described in enabling detail below.

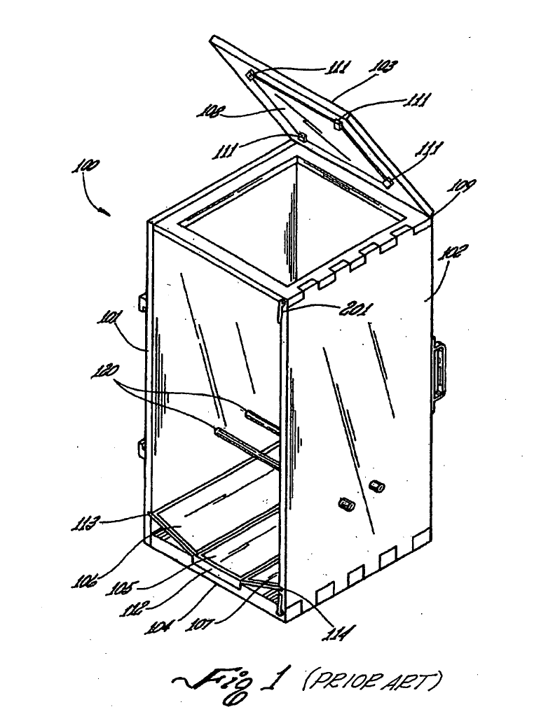

Briefly, the aforesaid prior art apparatus for the self-inspection of a person's feet is illustrated in perspective view in FIG. 1. The apparatus 100 comprises a left vertical wall 101, a right vertical wall 102, a top wall 103, and a bottom wall 104. Attached to bottom wall 104 is bottom mirror 105, side mirror 1 106, and side mirror 2 107. Top mirror 108 is affixed to top wall 103 by affixing means 111. Foot rests are provided by support rods 120. Top wall 103 is affixed to left vertical wall 101 and right vertical wall 102 via hinging means 109. Hinging means 109 enables top wall 103 along with attendant top mirror 108 to rotate about an axis. Hinging means 109 is preferably able to hold into place both top mirror 108 and top wall 103 in a stationary position along any angle which is most convenient for user. Or in the alternative, an elongated member may also be affixed to the top edge of either the left vertical wall 101 or right vertical wall 102 to hold the top wall 103 at a desired angle for viewing. In operation, a person seats himself such that he/she is facing the top mirror 108 and places his/her feet on the support rods 120. The seated person is then able to view the soles and sides of their feet through the top mirror 108. The top mirror 108, in turn, receives the image of the feet through light reflected from the bottom mirror 105 and the side mirrors 106 and 107. Bottom mirror 105, which may be a magnifying mirror, reflects the image of the feet (not shown) to the top mirror 108 via side mirrors 106 and 107. Side mirrors 106 and 107 are angled in such a fashion such that they face each other. While the prior art apparatus 100 may be partially disassembled for transport or storage, disassembly and reassembly are time consuming. Accordingly, the present inventor has improved his prior apparatus 100 to include means for rapidly extending the apparatus for its intended use and for collapsing the apparatus for compact storage and/or transport when not in use.

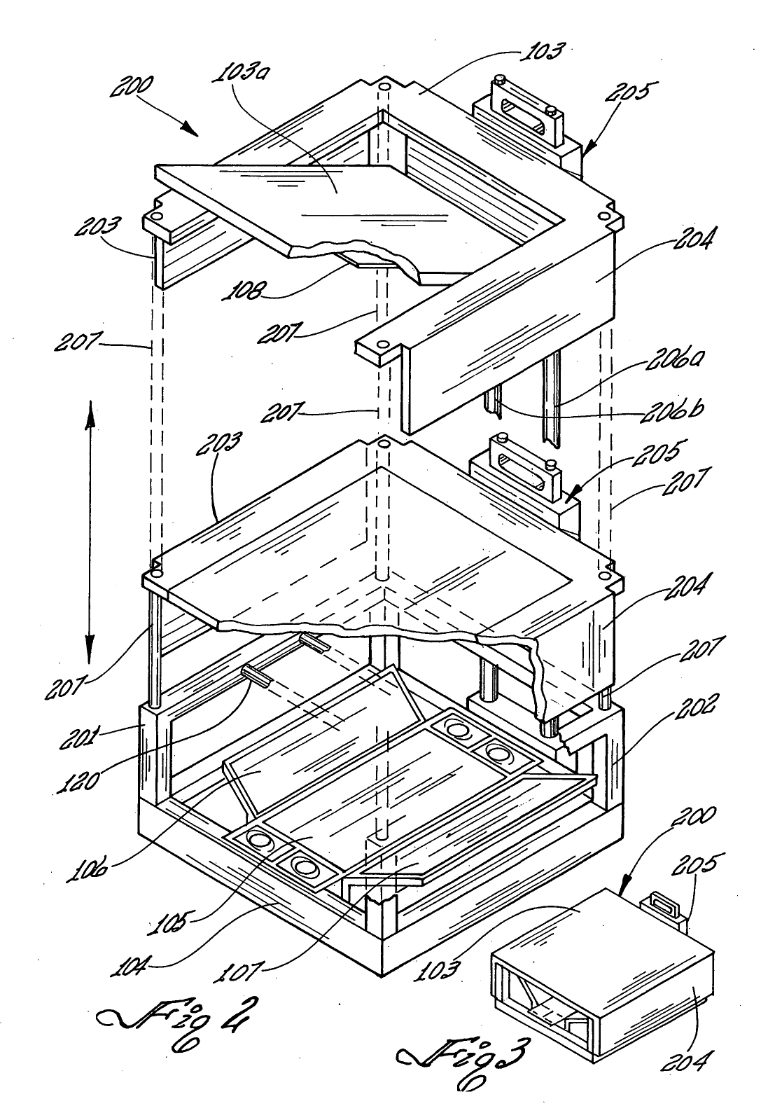

Turning now to FIG. 2, an improved, telescopically extendable apparatus operable for enabling the self-inspection of a person's feet is illustrated in perspective view at numeral 200. The construction of the apparatus 200 is similar to the construction and operation of the prior art apparatus 100 (FIG. 1) except that the left vertical wall 101 and the right vertical wall 102 of the prior art apparatus 100 (FIG. 1) are horizontally bifurcated and have lower portions 201 and 202 respectively and upper portions 203 and 204 respectively. The upper portion 203 of the left vertical wall and the upper portion 204 of the right vertical wall are both attached to the top wall 103. The top wall 103 has a hinged portion 103a that supports the top mirror 108. A lockable extendable member 205, such as an extendable handle commonly used with luggage, connects the top wall 103 to the bottom wall 104 of the apparatus 200. The extendable member 205 includes a locking and releasing mechanism and at least one telescopically extendable top support rod 206a and 206b (two extendable top support rods are shown in FIG. 2) that connects the bottom wall 104 to the top wall 103. The apparatus preferably includes telescopically extendable guide rods 207 at the corners of the bottom wall 104 that connect the bottom wall 104 to the top wall 103. Such guide rods 207 provide the apparatus 200 with additional stability when the extendable rods 206a and 206b are fully extended.

While particular embodiments of the present invention have been illustrated and described, it would be obvious to those skilled in the art that various other changes and modifications can be made without departing from the spirit and scope of the invention. It is therefore intended to cover in the appended claims all such changes and modifications that are within the scope of this invention.

What I claim is:

1. An apparatus for inspecting feet comprising a bifurcated left vertical wall, a bifurcated right vertical wall, a top wall having a top mirror tiltably attached thereto, a bottom wall having a left side mirror, a right side mirror and a center mirror attached thereto, and telescopically extendable locking means connecting said top wall and said bottom wall

2. The apparatus of claim 1 further comprising feet holding bars attached to a lower portion of said left and right bifurcated vertical walls.

3. The apparatus of claim 1 wherein said top wall and said bottom wall are connected to one another by telescopically extendable support rods.

4. The apparatus of claim 2 wherein said top wall and said bottom wall are connected to one another by telescopically extendable support rods.

5. An apparatus for inspecting feet comprising a top wall having a top mirror tiltably attached thereto, a bottom wall having a left side mirror, a right side mirror and a center mirror attached thereto, and telescopically extendable locking means connecting said top wall and said bottom wall.

6. The apparatus of claim 5 further comprising a plurality of telescopically extendable support rods connecting said top wall to said bottom wall, said support rods being operable for stabilizing said apparatus when said telescopically extendable locking means is in an extended and locked configuration.
